(19) United States
(12) Patent Application Publication
(10) Pub. No.: US 2016/0305965 A1
(43) Pub. Date: Oct. 20, 2016

(54) METHOD FOR DETERMINING THE HEMOSTATIC RISK OF A SUBJECT

(71) Applicant: KONINKLIJKE PHILIPS N.V., Eindhoven (NL)

(72) Inventors: BART JACOB BAKKER, EINDHOVEN (NL); HENDRIK JAN VAN OOIJEN, WIJK EN AALBURG (NL); RENE VAN DEN HAM, UTRECHT (NL)

(21) Appl. No.: 15/101,963

(22) PCT Filed: Dec. 10, 2014
(86) PCT No.: PCT/EP2014/077096

§ 371 Date: Jun. 6, 2016

(30) Foreign Application Priority Data
Dec. 19, 2013 (EP) 13198633.3

(51) Int. Cl. G01N 33/86

(52) U.S. Cl. CPC G01N 33/86; G01N 2800/50

(57) ABSTRACT

The present invention relates to clinical decision support systems. In detail, the present invention relates to a method for determining the hemostatic risk of a subject, to the use of a biomarker's threshold for determining the hemostatic risk of a subject, to a device for determining the hemostatic risk of a subject, to a computer program comprising a program code for carrying out the method for determining the hemostatic risk of a subject, and to a computer-readable non-transitory storage medium containing instructions for carrying out the method for determining the hemostatic risk of a subject.

### FIELD OF THE INVENTION

The present invention relates to clinical decision support systems. In detail, the present invention relates to a method for determining the hemostatic risk of a subject, to the use of a biomarker for determining the hemostatic risk of a subject, to a device for determining the hemostatic risk of a subject, to a computer program comprising a program code for carrying out the method for determining the hemostatic risk of a subject, and to a computer-readable non-transitory storage medium containing instructions for carrying out the method for determining the hemostatic risk of a subject.

### BACKGROUND OF THE INVENTION

Deep vein thrombosis is a wide spread problem in the western world (Kyrle P A, Eichinger S. Deep vein thrombosis. Lancet. 2005; 365(9465):1163-74). Large portions of the population run an increased risk of thrombosis, e.g. the elderly (Engbers M J, van Hylckama Vlieg A, Rosendaal F R. Venous thrombosis in the elderly: incidence, risk factors and risk groups. J Thromb Haemost. 2010; 8(10):2105-12), people who travel (Cannegieter S C, Doggen C J, van Houwelingen H C, Rosendaal F R. Travel-related venous thrombosis: results from a large population-based case control study (MEGA study). PLoS Med. 2006; 3(8):e307), and patients that undergo orthopaedic surgery (Kearon C, Kahn S R, Agnelli G, Goldhaber S, Raskob G E, Comerota A J; American College of Chest Physicians. Antithrombotic therapy for venous thromboembolic disease: American College of Chest Physicians Evidence-Based Clinical Practice Guidelines (8th Edition). Chest. 2008 June; 133(6 Suppl):4545-5455). People at risk can be put on preventive anticoagulant treatment, but the risk of bleeding (1-3% per year) (Veeger N J, Piersma-Wichers M, Tijssen J G, Hillege H L, van der Meer J. Individual time within target range in patients treated with vitamin K antagonists: main determinant of quality of anticoagulation and predictor of clinical outcome. A retrospective study of 2300 consecutive patients with venous thromboembolism. Br J Haematol. 2005; 128(4):513-9), and issues of cost and inconvenience (Cohen A T, Tapson V F, Bergmann J F, Goldhaber S Z, Kakkar A K, Deslandes B, Huang W, Zayaruzny M, Emery L, Anderson F A Jr; ENDORSE Investigators. Venous thromboembolism risk and prophylaxis in the acute hospital care setting (ENDORSE study): a multinational cross-sectional study. Lancet. 2008; 371(9610):387-94), speak against this. It would therefore be desirable to have a patient specific measure to estimate the personal thrombosis risk and facilitate an informed choice on whether or not to treat.

Unfortunately, with current clinical screening techniques and available methodologies, high risk individuals are not easily recognized and events are not accurately predicted (White R H. The epidemiology of venous thromboembolism. Circulation. 2003; 107(23 Suppl 1):14-8). One of the main reasons that this continues to be the case is that the vast majority of patients who suffer from thrombosis, those without obvious genetic defects, have blood coagulation systems that are not clinically identified as abnormal by routine screening tools and factor assays. Identification of individuals who are at risk for venous thrombosis is an area of research that could benefit from innovative technical methods (Brummel-Ziedins K E, Orfeo T, Rosendaal F R, Undas A, Rivard G E, Butenas S, Mann K G. Empirical and theoretical phenotypic discrimination. J Thromb Haemost. 2009; 7 (Suppl 1):181-6).

The coagulation system has been the topic of extensive research in the past century. The sub-system that has been studied in the most detail is the coagulation cascade. This cascade describes the coagulation process from exposure of the blood to tissue factor (a protein that is normally shielded beneath the vessel wall, but triggers coagulation when exposed) to the production of thrombin, a key protein in the clotting process. The production of thrombin is well captured in the Thrombin Generation Assay (TGA) (Hemker, H C and Béguin, S. Thrombin generation in plasma: Its assessment via the endogenous thrombin potential, Thrombosis and haemostasis, vol. 74, no. 1, pp. 134-138, 1995), which measures thrombin concentration over time, after addition of a known concentration of tissue factor to a blood sample. Several features of the TGA, like lag time (time between the tissue factor trigger and occurrence of the first non-zero concentrations of thrombin), maximum thrombin concentration, time to maximum, maximum generation rate and endogenous thrombin potential (ETP, area under the plotted thrombin curve over time) have been tentatively linked to thrombosis risk.

Research (Jordan, S W and Chaikov, E L. Simulated Surface-Induced Thrombin Generation in a Flow Field. Biophysical Journal, Volume 101, July 2011 276-286) has shown that thrombin generation grows stronger (higher TGA maximum, shorter lag times) with increasing concentrations of tissue factor. For low (˜1 fM) concentrations of tissue factor, no significant thrombin generation occurs, whereas for concentrations in the pM range it does. Based on this, the concept of a tissue factor threshold, i.e. a minimum concentration of tissue factor required for coagulation to start, has been hypothesized and observed (Mann, K G, Butenas, S. and Brummel, K. The Dynamics of Thrombin Formation. Arterioscler. Thromb. Vasc. Biol. 2003; 23:17-25, Jordan and Chaikov, l.c.).

Uncertainty about the patient specific risk of thrombosis causes unnecessary cases of thrombosis in patients at high risk (of thrombosis) who do not receive anticoagulant treatment. On the other hand, this uncertainty can result in bleeding in patients at relatively low risk who do receive unnecessary anticoagulant treatment. The current state of the art (Hippisley-Cox, J and Coupland, C. Development and validation of risk prediction algorithm (QThrombosis) to estimate future risk of venous thromboembolism: prospective cohort study, BMJ 2011; 343) estimates thrombosis risk based on a number of clinical risk factors. This is however not sufficiently specific.

Documents US2009/0298103 and WO 2009/142744 disclose a method for determining a hemostatic risk in a patient by subjecting the concentrations of various blood factors to a computer model. With this model the thrombin concentrations are simulated and the simulated concentrations are compared to a reference. According to the authors, the comparison allows a decision of a clinician whether the patient is predisposed to a hemostatic risk. However, such method has not proven its value in the clinical practice.

### SUMMARY OF THE INVENTION

It is an object of the invention to provide a method for determining the hemostatic risk of a subject by means of which the disadvantages of the prior art methods can be avoided. In particular, such a method for determining the hemostatic risk of a subject should be provided which allows a reliable diagnosis on whether a subject has a high risk of thrombosis and might require anti-coagulation medication or whether it has a high risk of bleeding contra-indicating or even requiring a stop of anti-coagulation medication.

It is another object of the invention to provide a device for determining the hemostatic risk of a subject, a computer program comprising a program code for carrying out a method for determining the hemostatic risk of a subject, and a computer-readable non-transitory storage medium containing instructions for carrying out a method for determining the hemostatic risk of a subject.

In a first aspect of the present invention a method for determining a hemostatic risk of a subject is presented, the method comprising a comparison of a concentration value of a clotting trigger sufficient to start the clotting process in a subject with at least one reference concentration value of said clotting trigger indicative for a hemostatic risk and/or a hemostatic non-risk (hemostatic stability).

In another aspect the present invention is directed to the use of a concentration value of a clotting trigger sufficient to start the clotting process in a subject for determining the hemostatic risk of said subject.

The method and use according to the invention can be realized in vitro, i.e. the physical presence of the subject, such as a living or human being, would not be necessary. For doing so, the concentration value of a clotting trigger sufficient to start the clotting process can be measured or generated in a step prior to the method or use according to the invention.

In still another aspect of the present invention a device is provided for determining the hemostatic risk of a subject, said device comprising:

a receiving unit configured for receiving a first information on the hemostatic condition of said subject,

a first determining unit configured for determining on the basis of said first information received by the receiving unit a concentration value of a clotting trigger sufficient to start the clotting process in said subject,

a comparing unit configured for comparing said concentration value determined by said first determining unit with at least one reference concentration value of said clotting trigger indicative for a hemostatic risk and/or a hemostatic non-risk (hemostatic stability).

In still another aspect of the present invention a clinical decision support system is presented, said system comprising a processor and a computer-readable storage medium, wherein said computer-readable storage medium contains instructions for execution by the processor, wherein said instructions cause said processor to perform the steps of:

a) receiving a first information on the hemostatic condition of a subject,

b) determining on the basis of said information of step (a) a concentration value of a clotting trigger sufficient to start the clotting process in said subject,

c) comparing said concentration value determined in step (b) with at least one reference concentration value of said clotting trigger indicative for a hemostatic risk and/or a hemostatic non-risk (hemostatic stability).

In yet further aspects of the present invention, there are provided a computer program which comprises program code means for causing a computer to perform the steps of the method disclosed herein when said computer program is carried out on a computer as well as a non-transitory computer-readable recording medium that stores therein a computer program product, which, when executed by a processor, causes the method disclosed herein to be performed.

Preferred embodiments of the invention are defined in the dependent claims. It shall be understood that the claimed system, device, computer program and medium have similar and/or identical preferred embodiments as the claimed method and as defined in the dependent claims. Therefore, all of the dependent claims referring to the method according to the invention can also be combined with the system, device, computer program and medium according to the invention and with each other.

The proposed invention significantly increases the accuracy of a person's specific thrombosis and bleeding risk estimation, especially within the increased risk subgroup of patients with at least one known clinical risk factor present. This subgroup involves—among others—patients that are hospitalized, are pregnant or are (start) using oral contraceptives and thus receive attention of a physician. In this context, the proposed invention helps the physician to stratify the patients that are treated or examined for conditions that are known to increase thrombosis risk, into high and low risk categories. Specifically, the improved method may be used to decide, per patient, whether or not to administer anticoagulant treatment based on estimated thrombosis or bleeding risk.

In contrast to the method known from prior art documents US2009/0298103 and WO 2009/142744 where thrombin as the key factor of the coagulation process is simulated in silico the invention does use a different approach. In the invention a clotting trigger is determined, i.e. a factor that initiates the coagulation process, rather than the coagulation process itself. This approach allows a more precise and reliable prediction of the hemostatic risk and does not necessitate a complex and error-prone simulation in silico of the thrombin formation like with the prior art method.

According to the invention “the hemostatic risk” refers to the risk of a subject, such as a human or animal being, of having a dysfunction of the haemostasis which might result in the development of thrombosis or bleeding.

According to a further development the method of the invention comprises the steps of:

a) providing a first information on the hemostatic condition of said subject,

b) determining on the basis of said first information of step (a) a concentration value of a clotting trigger sufficient to start the clotting process in said subject,

c) comparing said concentration value determined in step (b) with a reference concentration value of said clotting trigger representing the minimum concentration for a stable hemostatic condition, and

d) determining a high risk of thrombosis for said subject if said concentration value determined in step (b) is lower than said reference concentration value of said clotting trigger representing the minimum concentration for a stable hemostatic condition,

wherein preferably steps c) and d), additionally or alternatively, comprise the following steps:

c′) comparing said concentration value determined in step (b) with a reference concentration value of said clotting trigger representing the maximum concentration for a stable hemostatic condition, and

d′) determining a high risk of bleeding for said subject if said concentration value determined in step (b) is higher than said reference concentration value of said clotting trigger representing the maximum concentration for a stable hemostatic condition.

In still another aspect of the present invention a device is provided for determining the hemostatic risk of a subject, said device comprising:

a receiving unit configured for receiving a first information on the hemostatic condition of said subject,

a first determining unit configured for determining on the basis of said first information received by the receiving unit a concentration value of a clotting trigger sufficient to start the clotting process in said subject,

a comparing unit configured for comparing said concentration value determined by said first determining unit with a reference concentration value of said clotting trigger representing the minimum concentration for a stable hemostatic condition, and

a second determining unit configured for determining a high risk of thrombosis for said subject if said concentration value determined by said first determining unit is lower than said reference concentration value of said clotting trigger representing the minimum concentration for a stable hemostatic condition,

wherein preferably, additionally or alternatively,

said comparing unit is configured for comparing said concentration value determined by said first determining unit with a reference concentration value of said clotting trigger representing the maximum concentration for a stable hemostatic condition, and said second determining unit is configured for determining a high risk of bleeding for said subject if said concentration value determined by said first determining unit is higher than said reference concentration value of said clotting trigger representing the maximum concentration for a stable hemostatic condition.

In still another aspect of the present invention a clinical decision support system is presented, said system comprising a processor and a computer-readable storage medium, wherein said computer-readable storage medium contains instructions for execution by the processor, wherein said instructions cause said processor to perform the steps of:

a) receiving a first information on the hemostatic condition of a subject,

b) determining on the basis of said information of step (a) a concentration value of a clotting trigger sufficient to start the clotting process in said subject,

c) comparing said concentration value determined in step (b) with a reference concentration value of said clotting trigger representing the minimum concentration for a stable hemostatic condition, and

d) determining a high risk of thrombosis for said subject if said concentration value determined in step (b) is lower than said reference concentration value of said clotting trigger representing the minimum concentration for a stable hemostatic condition,

wherein preferably, steps c) and d) additionally or alternatively comprise the following steps:

c′) comparing said concentration value determined in step (b) with a reference concentration value of said clotting trigger representing the maximum concentration for a stable hemostatic condition, and

d′) determining a high risk of bleeding for said subject if said concentration value determined in step (b) is higher than said reference concentration value of said clotting trigger representing the maximum concentration for a stable hemostatic condition.

According to the invention a “first information on the hemostatic condition” refers to any information characterizing the hemostatic state of the subject, preferably the state before the clotting or coagulation process has been initiated, such as concentration values of (preferably inactivated) coagulation molecules or proteins, enzymatic constants, output from functional assays like the INR assay, or indirect information like genetic data on e.g. ‘Factor V Leiden’ that provides indirect information on how efficient the protein ‘Factor V’ can be inactivated by protein ‘activated protein C’. Such first information can be measured for said specific subject or average values taken from literature can be used or such information can be partly formed by both approaches.

According to the invention a “clotting trigger” refers to a factor being present in the subject's body liquid, such as blood, being capable of initiating the coagulation process in the subject. The coagulation process is initiated when the clotting trigger reaches a critical concentration in the subject's body liquid. According to the invention said critical concentration is referred to the “concentration value sufficient to start the clotting process in said subject” or “clotting trigger threshold”. Suitable clotting triggers encompass tissue factor (TF) (extrinsic pathway), but also platelet tissue factor, thromboplastin, or CD142, activated coagulation factors, e.g. FIIa, FVa, FVIIa, FVIIIa, FIXa, FXa, FXIa, FXIIa, collagen, high-molecular-weight kininogen (HMWK), prekallikrein and FXII or a combination thereof (intrinsic pathway) can be used as clotting triggers.

According to the invention in step (b) such critical concentration of the clotting trigger can be determined by measurements of the clotting or coagulation process as a function of the clotting trigger concentration in vitro, allowing an implementation in a laboratory environment. It can also be determined by employing a computer simulation which takes the information of the hemostatic condition of the subject, such as concentration values of coagulation proteins from the subject's blood sample (without the clotting trigger) as inputs, and calculating the clotting response depending on the clotting trigger concentrations.

A “reference concentration value of said clotting trigger” refers to a value of the clotting trigger which has been obtained from one or a plurality of reference subjects or reference in silico calculations. The reference concentration can be the result of an individual measurement or calculation or an average value of a plurality of measurements or calculations, respectively.

According to the invention the “minimum/maximum concentration for a stable hemostatic condition” refers to concentration values of the clotting trigger limiting a concentration range of said clotting trigger which can be found in healthy reference subjects characterized by a functional, stable hemostasis (‘healthy range’). The minimum concentration of the clotting trigger limits the healthy range at its lower boundary; the maximum concentration of the clotting trigger limits the healthy range at its upper boundary. Alternatively, said minimum and maximum concentrations might refer to concentration values of the clotting trigger limiting a concentration range of said clotting trigger which can be found in patient subjects on anticoagulants (‘therapeutic range’). Both ranges can be inferred from literature or determined in a targeted patient study.

Said “concentration value of a clotting trigger sufficient to start the clotting process” is also referred to as a “clotting trigger threshold”. For concentrations of the clotting trigger below that threshold the clotting response remains at a minimum, for concentrations of the clotting trigger above the threshold the clotting response starts to rise. In steps (c) and (c′) the clotting trigger threshold is compared with the boundary values of the ‘healthy range’.

According to the invention, if the clotting trigger threshold as determined in step (b) falls below the ‘healthy range’ the subject is deemed to be at high risk of thrombosis as diagnosed in step (d) and anti-coagulant treatment might be indicated. If the threshold falls above the range, the patient is deemed to be at high risk of serious bleeding as diagnosed in step (d′) and anti-coagulant use might be counter-indicated or may even be stopped if the patient was using anticoagulants at the time of testing.

The object underlying the invention is herewith fully achieved. The inventors have surprisingly realized that the concentration value of a clotting trigger, such as a concentration value of the tissue factor (TF), sufficient to start the clotting process in a subject can be used for determining the hemostatic risk of said subject. Even though the notion of a ‘tissue factor threshold’ exists and has been mentioned in literature the use of this threshold as a way to indicate thrombosis or bleeding risk is neither disclosed nor rendered obvious by the prior art. Furthermore, in the art the concept of a ‘tissue factor threshold’ has always been linked to visual signs of clotting, e.g. solidifying of the blood sample or thrombin generation. The use of other coagulation features, which often manifest themselves before thrombin generation or formation of the clot, is made possible by the method according to the invention and may provide an improvement over thrombin based methods in the art.

According to an alternative embodiment of the invention the method comprises the following steps:

a) providing a first information on the hemostatic condition of said subject (S1),

b) determining on the basis of said first information of step (a) a concentration value of a clotting trigger sufficient to start the clotting process in said subject (S2),

c) comparing said concentration value determined in step (b) with at least two or more reference concentration values of said clotting trigger comprising at least one reference concentration value indicative for a hemostatic risk, preferably a high risk of thrombosis and/or a high risk of bleeding, and at least one reference concentration value indicative for a hemostatic non-risk (hemostatic stability) (S3″), and

d) determining
a hemostatic risk for said subject if said concentration value determined in step (b) is numerically closer to said at least one reference concentration value indicative for a hemostatic risk (S4″), or
a hemostatic non-risk (hemostatic stability) for said subject if said concentration value determined in step (b) is numerically closer to said at least one reference concentration value indicative for a hemostatic stability (S4″).

In this alternative approach results the clotting trigger threshold is compared with (different) reference values in view of being numerically closest to one of the latter. This results in a gliding scale rather than a binary output. It is preferred that steps (c) and (d) are realized by means of a nearest neighbor approach or a nearest-neighbor interpolation, respectively.

According to that alternative embodiment of the invention a device for determining the hemostatic risk of a subject is provided, said device comprises:

a receiving unit configured for receiving a first information on the hemostatic condition of said subject,

a first determining unit configured for determining on the basis of said first information received by the receiving unit a concentration value of a clotting trigger sufficient to start the clotting process in said subject,

a comparing unit configured for comparing said concentration value determined by said first determining unit with at least two or more reference concentration values of said clotting trigger comprising at least one reference concentration value indicative for a hemostatic risk, preferably a high risk of thrombosis and/or a high risk of bleeding, and at least one reference concentration value indicative for a hemostatic non-risk (hemostatic stability), and

a second determining unit configured for determining
a hemostatic risk for said subject if said concentration value determined by said first determining unit is numerically closer to said at least one reference concentration value indicative for a hemostatic risk, or
a hemostatic non-risk (hemostatic stability) for said subject if said concentration value determined by said first determining unit is numerically closer to said at least one reference concentration value indicative for a hemostatic stability. Consequently, in another aspect of the invention a clinical decision support system is provided comprising a processor and a computer-readable storage medium, wherein said computer-readable storage medium contains instructions for execution by the processor, wherein said instructions cause said processor to perform the steps of:

a) providing a first information on the hemostatic condition of said subject,

b) determining on the basis of said first information of step (a) a concentration value of a clotting trigger sufficient to start the clotting process in said subject,

c) comparing said concentration value determined in step (b) with at least two or more reference concentration values of said clotting trigger comprising at least one reference concentration value indicative for a hemostatic risk, preferably a high risk of thrombosis and/or a high risk of bleeding, and at least one reference concentration value indicative for a hemostatic non-risk (hemostatic stability), and

d) determining
a hemostatic risk for said subject if said concentration value determined in step (b) is numerically closer to said at least one reference concentration value indicative for a hemostatic risk, or
a hemostatic non-risk (hemostatic stability) for said subject if said concentration value determined in step (b) is numerically closer to said at least one reference concentration value indicative for a hemostatic stability.

According to a further aspect, in step (a) said first information on the hemostatic condition of said subject is a concentration value of a coagulation protein in a biological sample from said subject, preferably in step (a) said first information on the hemostatic condition of said subject is the concentration values of a plurality of coagulation proteins in a biological sample from said subject, highly preferably in step (a) said first information on the hemostatic condition of said subject is the concentration values of at least three or more coagulation proteins in a biological sample from said subject.

These measures have the advantage that such information on the hemostatic condition of a subject is used for realizing the method according to the invention which has been proven as being appropriate for the determination of the clotting trigger threshold. Preferably, in this embodiment the coagulation proteins are present in their inactivated form. The concentration values of the coagulation proteins can be measured for said specific subject or average values taken from literature can be used. Alternatively or additionally, the concentration values measured for said specific subject and combined with the average values taken from literature for those coagulation proteins which are not measured in said specific subject. A “biological sample” refers to any biological material of the subject, such as biological cells such as endothelial cells, biological tissue such as the endothelium, or preferably biological liquids such as blood.

According to another aspect said clotting trigger is the tissue factor (TF).

This measure has the advantage that such a clotting trigger is employed which is thought to be the main trigger of coagulation and has been proven as being particularly suited for the realization of the invention.

According to a further aspect said coagulation protein(s) is/are selected from the group consisting of: coagulation factor 2 (FII), FV, FVII, FVIII, FIX, FX, FXI, FXII, antithrombin (AT), TFPI, α2M, C4BP, protein C, protein S, protein Z, TAFI, ZPI, AAT, PCI, C1 inhibitor and fibrinogen.

This further development of the invention has the advantage that such (inactivated) coagulation proteins are used to calculate the clotting trigger threshold which, according to the findings of the inventors, produce notably good and reliable results.

Pursuant to another preferred embodiment of the invention in step (b) said concentration value of a clotting trigger sufficient to start the clotting process in the subject is determined by an in silico simulation of the clotting process.

The clotting trigger could be determined in situ or vitro, e.g. by visually examining signs of clotting or solidifying of the blood sample, respectively, or thrombin generation. The use of other coagulation features, which often manifest themselves before thrombin generation or formation of the clot, is made possible by the use of a computer model of the coagulation process. Such in silico simulation may provide an improvement over thrombin based methods.

According to another preferred embodiment in the in silico simulation said first information on the hemostatic condition of said subject, such as concentration values of (inactivated) coagulation proteins, is used as input feature, and a second information on the hemostatic condition of said subject in the simulated clotting process is used as output feature.

According to another preferred embodiment said second information on the hemostatic condition of said subject is the concentration values of an activated coagulation protein at a series of time points of the simulated clotting process which are used as a set of output features, preferably said second information on the hemostatic condition of said subject is the concentration values of a plurality of activated coagulation proteins at a series of time points of the simulated clotting process which are used as a set of output features. However, it is to be understood that, even the activated coagulation proteins are preferred, in principle the concentration values of non-activated coagulation proteins can be used as well. If, e.g., the concentration values of non-activated coagulation proteins are known over time, such as FX, also the concentration values of the produced activated counterpart are known, i.e. FXa, since this is the initial concentration of FX minus the concentrations of FX at later stages.

In comparison with a determination of the clotting trigger threshold in vitro or in situ a much wider range of features can be simulated in a computer simulation which takes measured protein concentrations from a patient's sample, such as a blood sample (without tissue factor trigger) and/or average values from literature as inputs and calculates the clotting response and the clotting trigger threshold, respectively. Therefore, such measure significantly increases the accuracy of the diagnosis of the subject's specific risk of thrombosis or bleeding.

According to a further development of the invention said activated coagulation protein(s) is/are selected from the group consisting of: thrombin (FIIa), FVa, FVIIa, FVIIIa, FIXa, FXa, FXIIa, FVa-FXa, FVIIIa-FIXa, fibrin, prothrombin (FII).

This measure has the advantage that any of the afore-mentioned activated coagulation proteins has been proven as being suitable to determine the clotting trigger threshold.

Said second information on the hemostatic condition of said subject can also be embodied by the endogenous thrombin potential (ETP). The ETP is defined as the time integral or the area under the curve of the thrombin concentration over the course of the simulation and is often related to the amount of fibrin which can be generated after the in vitro activation of coagulation with tissue factor as trigger and phospho lipids as platelet substitute.

According to another aspect of the invention out of the set of output features one feature is created representing the strength of the clotting response, preferably said one feature representing the strength of the clotting response is the maximum concentration of at least one of said activated coagulation proteins over all time points of the simulated clotting process or the ETP (time integral over an activated protein (e.g. FIIa) or total production of thrombin (i.e. FII(t=0)−FII(t=t_end), where t=0 denotes the time of first exposure to a clotting trigger and t_end denotes that time after first exposure (e.g. one hour) that is deemed to be such that the clotting process takes place completely within the interval t=0 to t=t_end).

This measure has the advantage that a feature is used which allows the in silico determination of the clotting trigger threshold. In the simulation the clotting response e.g. the maximum concentration of a specific activated coagulation protein, such as F10a, is used, or a variety of activated coagulation proteins, to calculate the concentration value of the clotting trigger, e.g. of TF, sufficient to start the clotting process.

It shall be understood that a preferred embodiment of the invention can also be any combination of the dependent claims with the respective independent claim.

These and other aspects of the invention will be apparent from and elucidated with reference to the embodiments described hereinafter.

### BRIEF DESCRIPTION OF THE DRAWINGS

In the drawings:

FIG. 1 shows a graph demonstrating the phenomenon of a clotting trigger threshold exemplified for the tissue factor;

FIG. 2 shows a model output of concentrations of an activated coagulation protein, exemplified for F10a, at 100 points in time during the simulated coagulation or clotting process;

FIG. 3 schematically shows a respective flow diagram of a method according to exemplary embodiments of the invention;

FIG. 4 schematically shows a respective flow diagram of a method according to another exemplary embodiment of the invention

FIG. 5 shows a bar diagram of the risk score evaluated on the basis of clinical risk factors (CRF), single nucleotide polymorphisms (SNPs), protein levels and model based thresholds in a cross-validation study on the MEGA database.

### DETAILED DESCRIPTION OF EMBODIMENTS

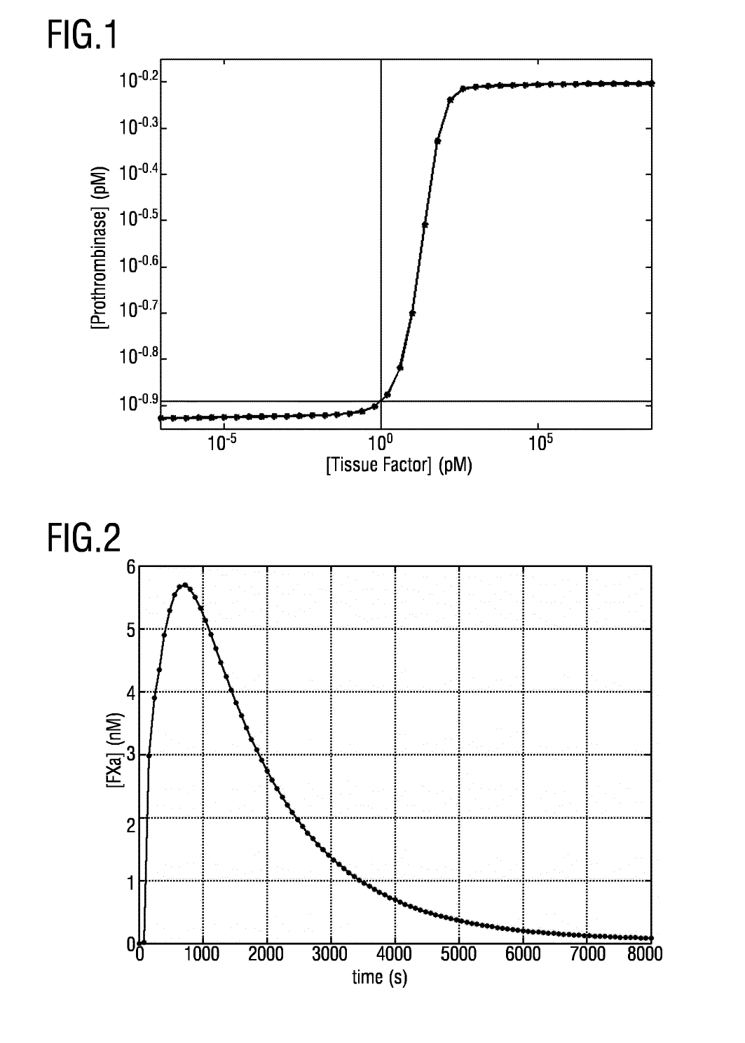

The invention identifies a person's expected clotting response at a series of clotting triggers, e.g. in the form of increasing concentrations of tissue factor, e.g. the main protein that initiates the clotting process. Reference is made to FIG. 1 giving an example for the phenomenon of a clotting trigger threshold exemplified for the tissue factor. Each star in the curve represents a computer simulation. The input of each simulation consists of the measured coagulation protein concentrations in a patient's blood sample, along with literature values for the unmeasured concentrations. The x-axis represents the concentrations of tissue factor that are used as the final model input. The y-axis represents one chosen feature from the simulations' output, in this case the maximum concentration of prothrombinase (FVa-FXa). It can be seen that for low concentrations of tissue factor the clotting response remains at a minimum. At the tissue factor threshold, indicated by the vertical line, the clotting response starts to rise visibly and the chosen model output feature exceeds the chosen start-off level, indicated by the horizontal line. For sufficiently large tissue factor concentrations the response levels off again.

The clotting response (the y-axis of FIG. 1) can be taken as any representative feature in the clotting process. Commonly studied features (in a research setting) are thrombin generation features like maximum thrombin concentration or lag time. Similar features can be used on the generation of other enzymes in the coagulation cascade like FXa or enzyme complexes like FVIIIa-FIXa. In a broader sense features like clot size can be used as well. Some such features can be measured in vitro, allowing an implementation in a laboratory environment. A much wider range of features can be simulated in a computer model which takes measured protein concentrations from a patient's blood sample (without tissue factor trigger) as inputs and calculated the clotting response for the aforementioned series of increasing tissue factor concentrations.

The numerical value of the tissue factor threshold (a concentration value) is recorded for a patient and compared to a pre-defined ‘healthy range’ or ‘therapeutic range’ for patients on anticoagulants. If the threshold falls below this range, the patient is deemed to be at high risk of thrombosis, and anti-coagulant treatment is indicated. If the threshold falls above the range, the patient is deemed to be at high risk of serious bleeding and anti-coagulant use is counter-indicated or may even be stopped (if the patient was using anticoagulants at the time of testing).

One embodiment of the invention involves the use of a computer model that is able to simulate the dynamic change in coagulation protein concentrations after exposure to tissue factor. The patient specific input of the model consists of concentration values of the proteins that play a role in the coagulation cascade before clotting. The model simulates a blood sample with these concentrations and a non-zero concentration of clotting trigger, e.g. tissue factor, at time t=0. The output of the model consists of the concentrations of all proteins and protein complexes that are involved in the coagulation process, for a series of time points tᵢ>0.

One simulation thus generates P*T numerical outputs, with P the number of proteins and protein c complexes that are described in the model and T the number of time points for which a model output is generated. Out of this set of outputs one feature is created that represents the strength of the coagulation response. An example of such a feature could be the maximum concentration of one protein, e.g. FXa, over all time points in the model output.

Reference is made to FIG. 2 where the model output FXa concentration at 100 points in time (0-8000 seconds in 80 second intervals) is shown. The maximum concentration (5.7 nM in this graph) can be used as coagulation response feature. Note that the concentration features of the proteins in the coagulation cascade are strongly correlated, so many different model output features, or combinations of model output features can be selected as the coagulation response feature to be used in the determination of the tissue factor threshold.

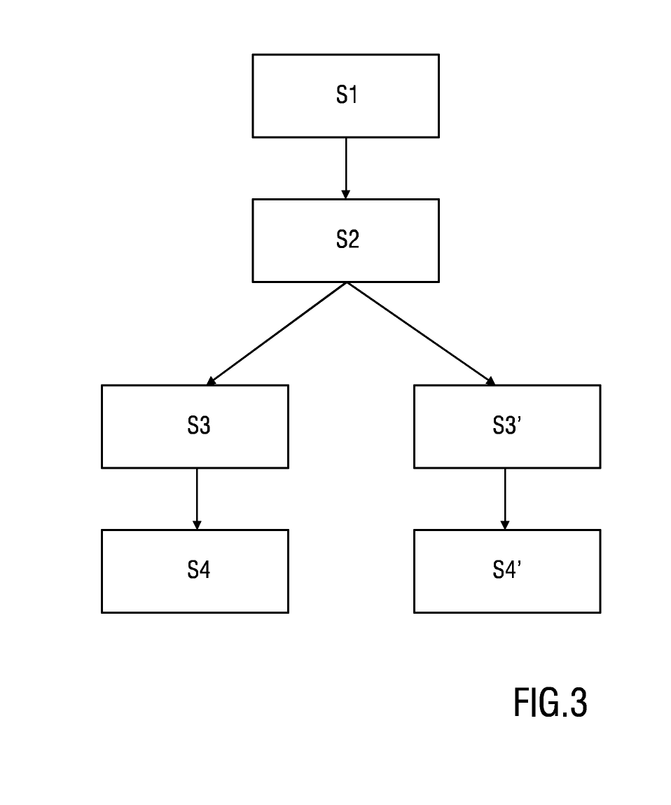

FIG. 3 shows a flow diagram of an embodiment of the method for determining the hemostatic risk of a subject wherein the presented method comprises steps (S1) to (S4) and/or (S1) to (S4′), respectively.

In step (S1) a first information on the hemostatic condition of said subject is provided, such as concentration values of a plurality of coagulation proteins in a biological sample from said subject. Such information may be received by a receiving unit of a device for determining the hemostatic risk of a subject or a clinical decision support system. The concentration values may be measured values or average values taken from literature. In practice, a blood sample, e.g. finger prick sample, is taken from a person for whom the hemostatic risk, i.e. the risk of thrombosis or bleeding needs to be determined. The concentrations of a number of (inactivated) coagulation proteins are determined in this sample via standard methods like ELISA assays. The measured proteins are preferably one, more or all of the following: coagulation factor 2 (FII), FV, FVII, FVIII, FIX, FX, FXI, FXII, antithrombin (AT), TFPI, α2M, C4BP, protein C, protein S, protein Z, TAFI, ZPI, AAT, PCI, Cl inhibitor and fibrinogen. In practice, the measurement of one or a subset (e.g. FII, FV, FVIII, FX, FXI, AT and fibrinogen) would be sufficient. Alternatively or in addition average values are taken from literature.

In step (2), on the basis of said first information of step (S1), a concentration value of a clotting trigger sufficient to start the clotting process in said subject is determined. Such determination might be realized by a determining unit of a device for determining the hemostatic risk of a subject or a clinical decision support system. In practice a computer model is used. With this model N simulations are performed with the following inputs: For the proteins in the measured (sub) set, use the measured values. For the unobserved protein concentrations, use the literature or average values. These inputs, i.e. the concentrations of the P proteins at time zero (the time when the simulated blood is first exposed to a clotting trigger), are the same in each of the N simulations. The concentration for the tissue factor varies between TFₘᵢₙ for the first simulation and TFₘₐₓ for simulation N. For every different choice of tissue factor concentration, a response is calculated through the model. In other words, a model simulation is performed which describes the coagulation response that corresponds to the chosen tissue factor concentrations. A response consists of the development of the concentration of a certain protein over time, starting at the time of first exposure of the blood to the clotting factor, e.g. tissue factor (initiation of the wound), and ending at a preset length of time. The model calculates these dynamics for a number of different proteins; FIG. 2 is an example for such protein where the response is limited to one characteristic quantity, e.g. the maximum concentration of FXa (the peak of the graph in FIG. 2). The concentration for simulation i may be chosen as

TFᵢ=TFₘᵢₙ+i*(TFₘₐₓ−TFₘᵢₙ)/N, (1)

or

TFᵢ=exp(log(TFₘᵢₙ₎+i*(log(TFₘₐₓ)−log(TFₘᵢₙ))/N) (2)

for i=0 . . . N, where formula (1) is most suitable when TFₘᵢₙ and TFₘₐₓ are of the same order of magnitude, whereas formula (2) is more suitable for larger ranges of tissue factor concentrations. Example values are TFₘᵢₙ=1×10⁻⁴ fM and TFₘₐₓ=1 mM, with N=100. This large range (for which formula (2) is the most fitting) is certain to include the tissue factor threshold in all but the most extreme cases, and N is high enough to catch its value with high precision.

As such, every combination of protein concentrations and clotting trigger or tissue factor concentration (as in the first item), can be used to calculate one numerical feature (one number) through the model. Then these numerical features are calculated for N different tissue factor concentrations, whereas the proteins that were measured in the patient or taken from literature were kept constant (the same values in each of the N model simulations). This produces N numerical features; FIG. 1 plots the value of one such feature on the Y-axis with the value for the corresponding tissue factor concentrations on the X-axis. The different values for the clotting trigger or tissue factor concentrations, respectively, start at a very low value, and increase with a certain step size up to a large value, such as to cover the range from the point where certainly no strong clotting response will take place, to the point where for sure a clotting response will take place. Formulae (1) and (2) describe these steps: Formula (1) describes a clotting trigger concentration exemplified for the minimum tissue factor concentration of TFₘᵢₙ and the maximum tissue factor concentration of TFₘₐₓ, with equal sized steps in between. The TFᵢ are the tissue factor concentrations for which the model output is calculated. Formula (2) describes the same, only now the steps have equal size in the log domain (for example, if the regarded tissue factor concentrations would vary over multiple orders of magnitude, say between 1 and 1.000.000, it would be more reasonable to look at tissue factor concentrations of 1, 10, 100, . . . , 1.000.000 than 1, 2, 3, . . . , 1.000.000; the first type of steps is what one gets in the log10 domain).

Once the model output is calculated for all clotting trigger or tissue factor concentrations, respectively, one will have a graph like in FIG. 1. By eye one can see where the response starts to rise. This point is referred to as the clotting trigger threshold or the tissue factor threshold, respectively. Such threshold can be calculated by the following algorithm:
Take the minimum Y-value in the graph of FIG. 1 [min(Yᵢ)] and the dynamic range, i.e. the difference between the highest and the lowest point in the graph [max(Yᵢ)−min(Yᵢ)]. It is hypnotized that the response starts to rise when it is more than 5% of the dynamic range higher than the minimum, leading to the formula:

min(Yᵢ)+0.05*(max(Yᵢ)−min(Yᵢ)) (3)
where Yᵢ is the obtained value for the coagulation response feature in simulation i;
where min(Yᵢ) is the minimum concentration value of the activated coagulation protein over all simulations, and
where max(Yᵢ) is the maximum concentration value of the activated coagulation protein over all simulations.

The horizontal line in FIG. 1 is an example of such a start-off level. An interpolation technique (e.g. linear) is used to determine the tissue factor threshold, i.e. the concentration where the coagulation response curve exceeds the start-off level (see vertical line in FIG. 1). The corresponding tissue factor threshold value for this patient is stored.

In step (S3) said concentration value or tissue factor threshold value, respectively, determined in step (S2) is compared with a reference concentration value of said clotting trigger representing the minimum concentration for a stable hemostatic condition. In addition or alternatively, in step (S3′) said concentration value determined in step (S2) is compared with a reference concentration value of said clotting trigger representing the maximum concentration for a stable hemostatic condition. Such comparison might be realized by a comparing unit of a device for determining the hemostatic risk of a subject or a clinical decision support system. In practice, in (S3) the tissue factor threshold is compared to a ‘minimum stable level’ (to be determined in a targeted patient study). In addition or alternatively, in (S3′) the tissue factor threshold can be compared to a ‘maximum stable level’. The ‘minimum stable level’ and ‘maximum stable level’ can be taken from literature or determined in a targeted patients study.

In step (S4) a high risk of thrombosis for said subject is determined if said concentration value determined in step (S2) is lower than said reference concentration value of said clotting trigger representing the minimum concentration for a stable hemostatic condition. In step (S4′) a high risk of bleeding for said subject is determined if said concentration value determined in step (S2) is higher than said reference concentration value of said clotting trigger representing the maximum concentration for a stable hemostatic condition. Such determining step might be realized by a determining unit of a device for determining the hemostatic risk of a subject or a clinical decision support system. In practice, if the patient's threshold is lower than the ‘minimum stable level’ level, in (S4) the patient is stratified as being ‘at high risk of thrombosis’. If the patient's threshold exceeds this level, in (S4′) the patient is stratified as being ‘at high risk of bleeding’. The combination of the minimum and maximum level can indicate a stable region, which may have different boundaries for anti-coagulant users (therapeutic region) and non-coagulant users (healthy region). Based on the risk stratification in (S4) and/or (S4′), the clinician can decide whether or not to start anticoagulant treatment on a non-anticoagulated patient, or to stop anticoagulant treatment on a patient who does currently use anticoagulants.

Steps (S1) and (S2) can be replaced by in vitro measurements of a coagulation response for a series of increasing clotting trigger or tissue factor concentrations, respectively. This is however more expensive in terms of time (and in most cases money), limits the choice of coagulation response feature and such measurements are known to become highly unreliable for small clotting triggers, e.g. ˜1 fM tissue factor. The advantage is that this embodiment does not rely on the quality of a computer model.

Furthermore, steps (S4) and/or (S4′) may be replaced by a data-driven algorithm. Such an algorithm can be a neural network based algorithm, which combines known thrombosis risk factors such as recent surgery, immobilization or the FV Leiden genetic mutation with the value for the clotting trigger or tissue factor threshold, respectively, that was obtained in step (S2), and returns a thrombosis risk score between zero and one. A similar algorithm can be described for bleeding risk.

Steps (S1), (S2), (S3), and (S4) and (S1), (S2), (S3′), and (S4′) can be seen as alternative routes. In case the risk of thrombosis is to be determined the first route (‘thrombosis risk route’; left route in FIG. 3) is used. In case the risk of bleeding is to be determined the second route (‘bleeding risk route’, right route in FIG. 3) is used. However, steps (S3) and (S3′) as well as steps (S4) and (S4′) can be taken in parallel or additionally, i.e. in one method, allowing the subsequent or parallel determination of the subject's risk of thrombosis or bleeding, e.g. by one device. It is also possible to go through the ‘thrombosis risk route’ and in case of a negative outcome to successively go through the ‘bleeding risk route’, i.e. (S1), (S2), (S3), (S4), (S3′), (S4′), or vice versa, i.e. (S1), (S2), (S3′), (S4′), (S3), (S4).

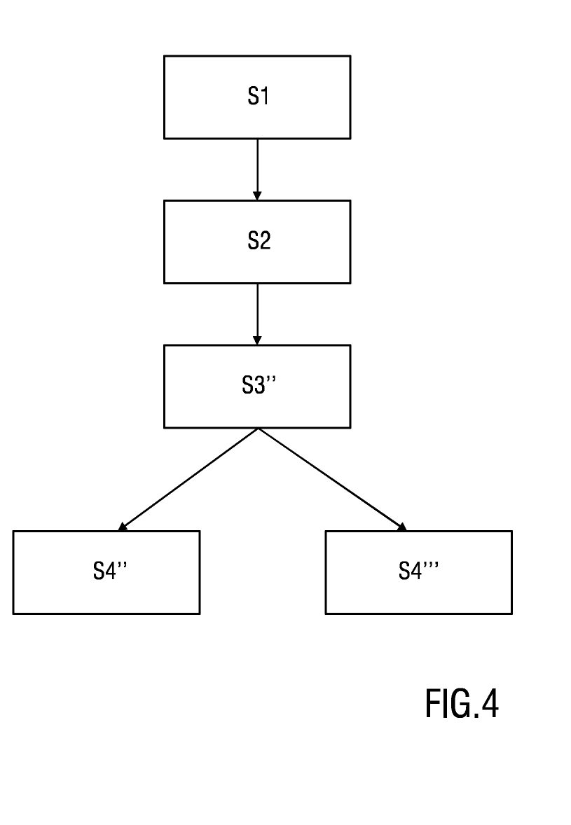

An alternative approach is depicted in FIG. 4. Steps (S1) and (S2) are identical with the approach shown in FIG. 3. However, in the next step (S3″) said concentration value determined in step (S2) is compared with at least two or more reference concentration values of said clotting trigger comprising at least one reference concentration value indicative for a hemostatic risk, preferably a high risk of thrombosis and/or a high risk of bleeding, and at least one reference concentration value indicative for a hemostatic non-risk (hemostatic stability). In step (S4″) a hemostatic risk for said subject is determined if said concentration value determined in step (S2) is numerically closer to said at least one reference concentration value indicative for a hemostatic risk. In contrast, a hemostatic non-risk (hemostatic stability) for said subject is determined if said concentration value determined in step (S2) is numerically closer to said at least one reference concentration value indicative for a hemostatic stability (S4′). Steps (S3″), (S4″), and (S4′) are realized by means of the nearest neighbor approach.

The inventors had the opportunity to evaluate the risk of deep vein thrombosis by using information from the MEGA (Multiple Environment and Genetic Assessment of risk factors for venous thrombosis) study. This is a case-control study that was set up to identify risk factors for venous thrombosis that have been performed in the Netherlands. A plethora of variables, ranging from coagulation protein levels to environmental thrombotic risk factors, to age and education level, and genetic thrombophilia has been taken from patients with venous thrombosis and controls. For the purpose of the present study, the inventors used the coagulation protein levels that were measured in the MEGA study to simulate the coagulation response for a range of tissue factor levels and identified the location of the tissue factor threshold for each patient. The inventors combined these model based risk factors with direct risk factors (i.e. clinical risk factors like recent surgery, genetic data and the numerical values of the same coagulation protein levels that are used in the model) in a regression method, to arrive at one thrombosis risk score for each patient. The identified combinatory risk score is validated in an internal cross-validation on the MEGA study.

MEGA: The MEGA study (Multiple Environmental and Genetic Assessment of risk factors for venous thrombosis study) is a large, population based case-control study on risk factors for venous thrombosis, of which details have been published previously. In brief, between March 1999 and September 2004, consecutive patients aged 18 to 70 years with a first objectively confirmed episode of deep venous thrombosis or pulmonary embolism were included from six participating anticoagulation clinics in the Netherlands. Information on the diagnostic procedure was obtained from hospital records and general practitioners. Only patients with a diagnosis of venous thrombosis that was confirmed with objective techniques were included in the analyses. Exclusion criteria were severe psychiatric problems and inability to speak Dutch. Of the 6567 eligible patients, 5184 participated (79%). For the present analysis, patients with arm vein thrombosis (n=228) and with pulmonary embolism were excluded (n=2069) to optimize the dataset with LETS in which only patients with DVT were included. As control persons, partners of patients aged <70 years without venous thrombosis were included (n=3277), as well as persons without venous thrombosis obtained via a random-digit-dialing (RDD) method (n=3000).

DATA COLLECTION: All persons were asked to complete an extensive questionnaire on many potential risk factors for venous thrombosis. Of particular interest for this study question are items on general health characteristics (age, sex, and immobilization). The index date was the date of the thrombotic event for patients and their partners, and the date of filling in the questionnaire for the random controls. The questionnaire also included questions about the presence of liver disease, kidney disease, rheumatoid arthritis, multiple sclerosis, heart failure, hemorrhagic stroke, and arterial thrombosis (myocardial infarction, angina, ischemic stroke, transient ischemic attack, and peripheral vascular disease) in the medical history.

For the current study the inventors selected the following risk factors: immobilization (plaster cast, extended bed rest at home for at least 4 days, hospitalization), surgery, a family history of venous thrombosis (considered positive if at least 1 parent, brother, or sister experienced venous thrombosis), leg injury in the past 3 months, cancer in the period from five years before to six month after the index date, travel for more than four hours in the past 2 months, pregnancy or puerperium within 3 months before the index date, or use of estrogens (oral contraceptives or hormone replacement therapy) at the index date. A further feature was the presence of obesity, determined as a body mass index of 30 kg/m² or higher.

GENETIC EFFECTS: Next to the data from the questionnaire, data was available on the presence of five genetic aspects, i.e. blood group non-O and four single nucleotide polymorphisms (SNPs) in F2 (rs1799963), fibrinogen (rs2066865), F11 (rs2036914) and F5 (FV Leiden; rs6025). The data further included the number of alleles that were affected per SNP.

BLOOD COLLECTION: Blood samples were taken at least 3 months after thrombosis was diagnosed. Whole blood (0.9 Vol.) was collected as previously described, from the antecubital vein into Sarstedt Monovette tubes (Nümbrecht, Germany) containing 0.106 M of trisodium citrate (0.1 Vol.). Plasma was prepared by centrifugation for 10 min at 2000 g at room temperature and stored in aliquots at −70° C. until assayed. All protein factor assays were previously performed and are either activity or antigen-based clinical assays. The included proteins are anti-thrombin (AT), prothrombin (factor II), factor 7 (FVII), FVIII, FIX, FX, FXI, fibrinogen and protein C (all activity measurements) and protein S (antigen measurement).

Individuals for whom no blood measurement was taken (1504 patients and 3357 controls) or less than 5 risk factors (protein levels, genetic effects and answers regarding the selected clinical risk factors) were available (n=1512 patients and n=3362) were excluded from this study. Patients that were on oral anticoagulant treatment at time of blood draw (n=294) and controls (n=34) were also excluded.

The final selection included 1227 patients and 2905 controls.

MODEL BASED RISK FACTORS—THE SENSITIVITY TO A CLOTTING TRIGGER INDICATES THROMBOSIS RISK: It is hypothesized that patients at increased risk of thrombosis start clotting at milder clotting triggers. The inventors hypothesize first that there is a threshold effect for the size of the clotting trigger. Taking the tissue factor trigger as an example, it is clear that no clotting should occur in the absence of tissue factor (no breach in the vessel wall). Very small yet non-zero tissue factor concentrations, corresponding to micro breaches of the vessel wall that occur regularly, should not lead to full blown coagulation. Serious breaches, corresponding to larger concentrations of tissue factor should however start a strong coagulation response, so somewhere between the two there should be a tissue factor threshold concentration where little to no coagulation changes into strong coagulation. The tissue factor concentration that corresponds to this threshold is taken as an indicative feature for thrombosis risk.

The coagulation response is calculated for a range of TF concentrations between 0.00004 fM and 40 nM. The range starts at an extremely low value to be sure to catch the first threshold; this threshold typically lies at TF concentrations higher than 0.4 fM and lower than 0.4 nM.

An example of the threshold effect is shown in FIG. 1, where the maximum concentration (or peak height (PH)) of FVa-FXa (prothrombinase) is plotted against the size of the simulated clotting trigger. The threshold concentration is indicated as that concentration where the selected feature (in this case FVa-FXa) starts to show a strong increase. Other features may be selected as well, like the time at which the maximum occurs (time to peak (TTP)), time until a protein concentration reaches 5% of its maximum (lag time), maximum rate of change in the concentration of a protein (max rate) or the area under the plotted curve (AUC). All of these features may be calculated for various proteins that play a role in coagulation. Table 1 shows a list of such features that are used to obtain the results that are described under RESULTS.

NEAREST NEIGHBOR APPROACH: To estimate whether a patient is at high or low risk according to one feature (e.g. the tissue factor threshold determined on the model output of maximum FVa-FXa concentration), it is to be proceeded as follows:

Calculate the feature for N subjects for whom the label (thrombosis/no thrombosis) is known

Calculate the same feature for the new patient

Collect the labels from the K subjects that have a predicted feature value that is numerically closest to the predicted feature for the new patient

Of the K collected labels, n_{case} will indicate thrombosis and n_{control} will not. The risk score for the new patient will now be calculated as [n_{case}/K]/Z, where Z is the fraction of thrombosis patients in the N subjects from step 1.

COMBINING BIOMARKERS: The next step is to combine the newly created biomarker features together, and with the earlier biomarker features: clinical risk factors, SNPs and protein concentrations. This rather large set of possibly predictive features is used as the input for data driven classification methods like neural networks. In the following the inventors applied a logistic regression based approach to infer the optimal risk score from the MEGA data. The scoring method is tested internally through a 500-fold cross-validation. Here, the inventors divided the 3866 participants randomly into a training set of 2577 participants and a non-overlapping test set of 1289 participants, under the constraint that both sets have the same ratio of cases to controls. The information in the training set, i.e. the aforementioned clinical risk factors, SNPs and protein levels for each subject, and a label (1/0) to indicate whether deep vein thrombosis (DVT) was diagnosed in a subject, is used to infer a risk score. This risk score is a numerical algorithm that takes numerical values for the patient features as inputs and produces a risk number between zero and one as output.

The inferred algorithm is subsequently used to calculate a risk number for each patient in the test set. The risk scores, along with the true subject case/control labels, are used to plot a ROC curve and the area under the curve (AUC) is a measure of the methods accuracy. This process is repeated 500 times, each time with a different random split of the data into training and test set. The average AUC and 95% confidence interval are presented to evaluate the method.

ROC CURVE: the proposed method assigns a risk score between zero and one for each subject in the study. If this score is compared to a number (threshold) between zero and one and thrombosis is predicted for those with a score above that threshold and no thrombosis for those below, a sensitivity (percentage of thrombosis patients for whom thrombosis is (correctly) predicted) and a specificity (1 minus the percentage of subjects without thrombosis for whom thrombosis is (erroneously) predicted) can be calculated. Depending on the thresholds, a range of combinations from 0% sensitivity with 100% specificity to 100% sensitivity with 0% specificity can be obtained. The ROC curve plots sensitivity on the y-axis and 1-specificity on the x-axis. The area under the curve (AUC) is often used as a quality measure of a risk score.

RESULTS: For calculation of the clotting trigger threshold the inventors considered the model output features as indicated in the left column of Table 1. The univariate AUCs (based on the ROC curve obtained using the related tissue factor threshold as the sole element in the risk score) given in the second column. Threshold features that scored 0.65 or more were found for total thrombin production (maximum rate), most FVIIIa-FIXa features, TFPI peak height, ETP and FII-FXa peak height.

TABLE 1
Model output features for the calculation of the clotting trigger.

| | |
|---|---|
| Total thrombin production | 0.66 |
| Maximum rate of total thrombin | 0.65 |
| FVa-FXa PH | 0.61 |
| FVa-FXa TTP | 0.61 |
| FVa-FXa lag | 0.58 |
| FVa-FXa max rate | 0.63 |
| FVa-FXa AUC | 0.62 |
| FVIIIa-FIXa PH | 0.67 |
| FVIIIa-FIXa TTP | 0.67 |
| FVIIIa-FIXa lag | 0.64 |
| FVIIIa-FIXa max rate | 0.67 |
| FVIIIa-FIXa AUC | 0.67 |
| TFPI PH | 0.66 |
| TFPI max rate | 0.63 |
| FIIa PH | 0.64 |
| FIIa TTP | 0.64 |
| FIIa lag | 0.61 |
| FIIa max rate | 0.63 |
| FIIa AUC | 0.65 |
| FVIII-FIIa PH | 0.59 |
| FVIII-FIIa TTP | 0.59 |
| FVIII-FIIa lag | 0.58 |
| FVIII-FIIa max rate | 0.60 |
| FVIII-FIIa AUC | 0.61 |
| FII-FXa PH | 0.68 |
| FII-FXa TTP | 0.63 |
| FII-FXa max rate | 0.63 |
| Fibrin TTP | 0.61 |
| Fibrin lag | 0.58 |

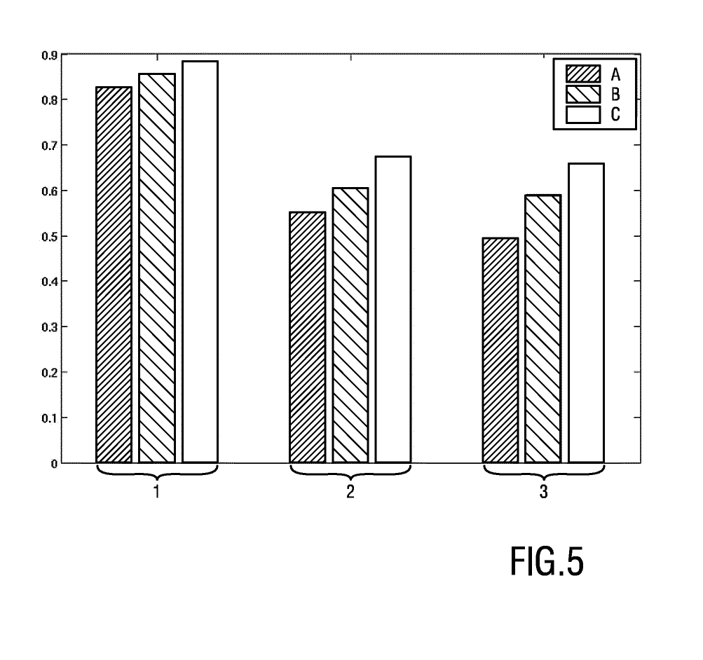

REGRESSION ON DIRECT FEATURES AND CLOTTING TRIGGER THRESHOLDS: Here a standard logistic regression function was used to evaluate the risk score based on clinical risk factors, SNPs, protein levels and model based thresholds in a cross validation study on the MEGA database. For the cross-validation the inventors make 500 random divisions of the data into a train and a validation set (2:1), where it was made sure that the case-control ratio is the same in both sets. For the threshold the inventors used a nearest neighbor score as described above, where all neighbors must be in the training set. The result is shown in FIG. 5 (A: CRF+SNP; B: CRF+SNP+Proteins; C: CRF+SNP+Proteins+Thresholds). The area under the ROC curve (FIG. 5, set of three bars on the left (1)), and correspondingly the maximum sensitivity that can be obtained in combination with 90% specificity (FIG. 5, set of three bars in the middle (2)) (and vice versa, the maximum specificity that can be obtained in combination with 90% sensitivity (FIG. 5, set of three bars on the right (3)) are significantly improved when tissue factor threshold features are included in the risk score. When the inventors compared the improvement due to inclusion of measured protein concentrations (from the bars to the very left to the bars in the middle of each set) to the total improvement (from the bars in the middle to the bars to the very right of each set), it can be seen that inclusion of the calculated tissue factor threshold, based on the exact same measured protein concentrations (i.e. requiring no additional measurement), doubles the increase in risk estimation accuracy.

SELECTION OF THE MOST INFORMATIVE FEATURES: The inventors performed a pruning method to identify the most relevant features. They proceeded as follows:

Making 500 divisions into ⅓ validation set and ⅔ train set

On the train set taking 100 random divisions into ⅓ test set and ⅔ (remains) train set

At each iteration removing, one by one, the feature that leads to the smallest decrease in estimation accuracy (AUC) on the training set. If multiple features cause the same (minimum) decrease in accuracy, pick one of these at random.

Store the number of times that each feature survives the pruning step described in the previous item, and the drop in accuracy caused by the removal of this feature.

After 100 iterations rank the features, first by number of pruning steps survived, second by average drop in accuracy upon removal.

Remove the features one by one in order of their ranking, and calculate the AUC on the 100 test sets.

For each of the 500 divisions, identify the minimum number of features that still leads to an average AUC on the test sets that exceeds the maximum average score on the test set minus one standard deviation (calculated around the maximum average score on the test set).

Count the number of times that each feature ends up in the remaining minimum feature set as described in the previous bullet point, over the 500 iterations.

On average 25 features end up in the selection made in the penultimate bullet point (95%CI=[19,31]). The ranking of the features that remain most often is shown in Table 2. It can be seen that the features that were most important in the previously published data driven approach (FVIII, FVLeiden, leg injury, etc.) still score high, but are now augmented in the top ten by two calculated threshold based features, i.e. the thresholds in total activated thrombin and endogenous thrombin potential.

TABLE 2
Ranking of the features most indicative for a hemostatic risk

| Feature name | Percentage selected |
|---|---|
| FVIII level | 100 |
| FV Leiden SNP | 100 |
| Leg injury | 100 |
| Recent operation | 100 |
| Family history | 100 |
| Oral contraceptive | 100 |
| use | |
| Immobility (hospital) | 100 |
| Immobility (home) | 100 |
| Free Protein S level | 100 |
| Fibrinogen SNP | 100 |
| TF threshold on tot. | 100 |
| FIIa | |
| TF threshold on ETP | 100 |
| Pregnancy | 100 |
| Gender | 100 |
| Plaster cast | 100 |
| Malignancies | 99 |
| FII SNP | 89 |
| TF threshold on | 86 |
| FVIIIa-FIXa max | |
| generation rate | |
| TF threshold on FVa- | 63 |
| FXa Peak Height | |
| TF threshold on | 62 |
| maximum FVIIIa- | |
| FIXa | |
| TF threshold on FVa- | 60 |
| FXa max generation | |
| rate | |
| Obesity | 58 |
| TF threshold on | 52 |
| maximum TFPI | |
| Blood group | 52 |

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable non-transitory medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. A method for determining a hemostatic risk of a subject, the method comprising a comparison of a concentration value of a clotting trigger sufficient to start the clotting process in a subject with at least one reference concentration value of said clotting trigger indicative for a hemostatic risk and/or a hemostatic non-risk (hemostatic stability), said method is comprising the steps of:
a) providing a first information on the hemostatic condition of said subject (S1),
b) determining on the basis of said first information of step (a) a concentration value of a clotting trigger sufficient to start the clotting process in said subject (S2),
c) comparing said concentration value determined in step (b) with a reference concentration value of said clotting trigger representing the minimum concentration for a stable hemostatic condition (S3), and
d) determining a high risk of thrombosis for said subject if said concentration value determined in step (b) is lower than said reference concentration value of said clotting trigger representing the minimum concentration for a stable hemostatic condition (S4),

2. The method of claim 1, wherein steps c) and d), additionally or alternatively, comprise the following steps:
c′) comparing said concentration value determined in step (b) with a reference concentration value of said clotting trigger representing the maximum concentration for a stable hemostatic condition (S3′), and
d′) determining a high risk of bleeding for said subject if said concentration value determined in step (b) is higher than said reference concentration value of said clotting trigger representing the maximum concentration for a stable hemostatic condition (S4′).

3. The method of claim 1 comprising the steps of:
a) providing a first information on the hemostatic condition of said subject (S1),
b) determining on the basis of said first information of step (a) a concentration value of a clotting trigger sufficient to start the clotting process in said subject (S2),
c) comparing said concentration value determined in step (b) with at least two or more reference concentration values of said clotting trigger comprising at least one reference concentration value indicative for a hemostatic risk, preferably a high risk of thrombosis and/or a high risk of bleeding, and at least one reference concentration value indicative for a hemostatic non-risk (hemostatic stability) (S3″), and
d) determining
a hemostatic risk for said subject if said concentration value determined in step (b) is numerically closer to said at least one reference concentration value indicative for a hemostatic risk (S4″), or
a hemostatic non-risk (hemostatic stability) for said subject if said concentration value determined in step (b) is numerically closer to said at least one reference concentration value indicative for a hemostatic stability (S4″),
wherein preferably steps (c) and (d) are realized by means of a nearest neighbor approach.

4. The method of claim 2, wherein in step (a) said first information on the hemostatic condition of said subject is a concentration value of a coagulation protein in a biological sample from said subject, preferably in step (a) said first information on the hemostatic condition of said subject is the concentration values of a plurality of coagulation proteins in a biological sample from said subject, highly preferably in step (a) said first information on the hemostatic condition of said subject is the concentration values of at least three or more coagulation proteins in a biological sample from said subject, wherein further preferably said coagulation protein(s) is/are selected from the group consisting of: coagulation factor 2 (FII), FV, FVII, FVIII, FIX, FX FXI, FXII, antithrombin (AT), TFPI, α2M, C4BP, protein C, protein S, protein Z, TAFI, ZPI, AAT, PCI, C1 inhibitor and fibrinogen.

5. The method of claim 2, wherein said clotting trigger is the tissue factor (TF).

6. (canceled)

7. The method of claim 2, wherein in step (b) said concentration value of a clotting trigger sufficient to start the clotting process in the subject is determined by an in silico simulation of the clotting process, wherein preferably in the in silico simulation said first information on the hemostatic condition of said subject is used as input feature, and a second information on the hemostatic condition of said subject in the simulated clotting process is used as output feature.

8. The method of claim 7, wherein said second information on the hemostatic condition of said subject is the concentration values of an activated coagulation protein at a series of time points of the simulated clotting process which are used as a set of output features, wherein preferably said second information on the hemostatic condition of said subject is the concentration values of a plurality of activated coagulation proteins at a series of time points of the simulated clotting process which are used as a set of output features, wherein most preferably said activated coagulation protein(s) is/are selected from the group consisting of: thrombin (FIIa), FVa, FVIIa, FVIIIa, FIXa, FXa, FXIIa, FVa-FXa, FVIIIa-FIXa, fibrin, prothrombin (FII).

9. The method of claim 8, wherein out of the set of output features one feature is created representing the strength of the clotting response, wherein preferably said one feature representing the strength of the clotting response is the maximum concentration of at least one of said activated coagulation proteins over all time points of the simulated clotting process.

10. Use of a concentration value of a clotting trigger, preferably of a concentration value of the tissue factor (TF), sufficient to start the clotting process in a subject for determining a hemostatic risk of said subject.

11. A device for determining a hemostatic risk of a subject, said device comprising:
a receiving unit configured for receiving a first information on the hemostatic condition of said subject,
a first determining unit configured for determining on the basis of said first information received by the receiving unit a concentration value of a clotting trigger sufficient to start the clotting process in said subject, and
a comparing unit configured for comparing said concentration value determined by said first determining unit with at least one reference concentration value of said clotting trigger indicative for a hemostatic risk and/or a hemostatic non-risk (hemostatic stability),
wherein said comparing unit is configured for comparing said concentration value determined by said first determining unit with a reference concentration value of said clotting trigger representing the minimum concentration for a stable hemostatic condition, and said device is further comprising:
a second determining unit configured for determining a high risk of thrombosis for said subject if said concentration value determined by said first determining unit is lower than said reference concentration value of said clotting trigger representing the minimum concentration for a stable hemostatic condition;
wherein preferably, additionally or alternatively,
said comparing unit is configured for comparing said concentration value determined by said first determining unit with a reference concentration value of said clotting trigger representing the maximum concentration for a stable hemostatic condition, and
said second determining unit is configured for determining a high risk of bleeding for said subject if said concentration value determined by said first determining unit is higher than said reference concentration value of said clotting trigger representing the maximum concentration for a stable hemostatic condition.

12. (canceled)

13. The device of claim 11, wherein said comparing unit is configured for comparing said concentration value determined by said first determining unit with at least two or more reference concentration values of said clotting trigger comprising at least one reference concentration value indicative for a hemostatic risk, preferably a high risk of thrombosis and/or a high risk of bleeding, and at least one reference concentration value indicative for a hemostatic non-risk (hemostatic stability), and said device is further comprising:
a second determining unit configured for determining:
a hemostatic risk for said subject if said concentration value determined by said first determining unit is numerically closer to said at least one reference concentration value indicative for a hemostatic risk, or
a hemostatic non-risk (hemostatic stability) for said subject if said concentration value determined by said first determining unit is numerically closer to said at least one reference concentration value indicative for a hemostatic stability.

14. A clinical decision support system comprising a processor and a computer-readable storage medium, wherein said computer-readable storage medium contains instructions for execution by the processor, wherein said instructions cause said processor to perform the steps of:
a) receiving a first information on the hemostatic condition of a subject,
b) determining on the basis of said information of step (a) a concentration value of a clotting trigger sufficient to start the clotting process in said subject,
c) comparing said concentration value determined in step (b) with at least one reference concentration value of said clotting trigger indicative for a hemostatic risk and/or a hemostatic non-risk (hemostatic stability),
wherein said instructions cause said processor to perform the steps of:
a) providing a first information on the hemostatic condition of said subject,
b) determining on the basis of said first information of step (a) a concentration value of a clotting trigger sufficient to start the clotting process in said subject,
c) comparing said concentration value determined in step (b) with a reference concentration value of said clotting trigger representing the minimum concentration for a stable hemostatic condition, and representing the minimum concentration for a stable hemostatic condition, and
d) determining a high risk of thrombosis for said subject if said concentration value determined in step (b) is lower than said reference concentration value of said clotting trigger representing the minimum concentration for a stable hemostatic condition,
wherein preferably, steps c) and d) additionally or alternatively comprise the following steps:
c′) comparing said concentration value determined in step (b) with a reference concentration value of said clotting trigger representing the maximum concentration for a stable hemostatic condition, and
d′) determining a high risk of bleeding for said subject if said concentration value determined in step (b) is higher than said reference concentration value of said clotting trigger representing the maximum concentration for a stable hemostatic condition.
wherein said instructions cause said processor to perform the steps of:
a) providing a first information on the hemostatic condition of said subject,
b) determining on the basis of said first information of step (a) a concentration value of a clotting trigger sufficient to start the clotting process in said subject,
c) comparing said concentration value determined in step (b) with at least two or more reference concentration values of said clotting trigger comprising at least one reference concentration value indicative for a hemostatic risk, preferably a high risk of thrombosis and/or a high risk of bleeding, and at least one reference concentration value indicative for a hemostatic non-risk (hemostatic stability), and
d) determining:
a hemostatic risk for said subject if said concentration value determined in step (b) is numerically closer to said at least one reference concentration value indicative for a hemostatic risk, or
a hemostatic non-risk (hemostatic stability) for said subject if said concentration value determined in step (b) is numerically closer to said at least one reference concentration value indicative for a hemostatic stability.

15. (canceled)

16. (canceled)

17. Computer program comprising program code means for causing a computer to carry out the steps of the method of claim 1 when said computer program is carried out on the computer.

18. A computer-readable non-transitory storage medium containing instructions for execution by a processor, wherein the instructions cause the processor to perform the steps of the method of claim 1.
